(19) United States
(12) Patent Application Publication
(10) Pub. No.: US 2026/0048243 A1
(43) Pub. Date: Feb. 19, 2026

(54) METHOD FOR PRODUCING DRAINAGE FOILS, TUBULAR DRAINAGE FOIL AND WOUND CARE KIT

(71) Applicant: Lohmann & Rauscher GmbH, Schönau/Triesting (AT)

(72) Inventors: Gunnar Loske, Ahrensburg (DE); Matthias Nemec, Kottingbrunn (AT); Christian Rohrer, Villach (AT); Erik Steinlechner, Baden (AT); Peter Grillitsch, Wien (AT)

(21) Appl. No.: 19/101,552

(22) PCT Filed: Aug. 11, 2023
(86) PCT No.: PCT/EP2023/072265

§ 371 Date: Feb. 5, 2025

(30) Foreign Application Priority Data
Aug. 12, 2022 (DE) 10 2022 120 423.5

(51) Int. Cl. A61M 27/00; A61L 31/04; A61M 25/00

(52) U.S. Cl. CPC A61M 27/00; A61L 31/048; A61M 2025/0004; A61M 2207/10

(57) ABSTRACT

A method for producing a double-walled drainage foil with an open drainage space comprising:
i) providing first and second web-shaped elements;
ii) perforating the first and second web-shaped elements, wherein in each of the first web-shaped element and the second web-shaped element, at least one opening allowing the passage of body fluids, preferably a multiplicity of openings allowing the passage of body fluids, is formed;
iii) arranging the first web-shaped element on the second web-shaped element in such a way that the first web-shaped element and the second web-shaped element are arranged approximately parallel;
iv) connecting the first web-shaped element and the second web-shaped element at at least one fastening region, in particular at at least one punctiform fastening region, preferably by ultrasonic welding, in order to obtain a double-walled drainage foil with an open drainage space formed between the first web-shaped element and the second web-shaped element.

### CROSS REFERENCE TO RELATED APPLICATION

The present application is a 35 U.S.C. § 371 national phase entry application of, and claims priority to, International Patent Application No. PCT/EP2023/072265, filed Aug. 11, 2023, which claims priority to German Patent Application No. DE 10 2022 120 423.5, filed Aug. 12, 2022, the disclosures of which are hereby incorporated by reference in their entirety for all purposes.

### TECHNICAL FIELD

Embodiments relate to a method for producing a double-walled drainage foil with an open drainage space and a method for producing a tubular drainage foil. Embodiments furthermore relate to a tubular drainage foil, a wound care kit and the tubular drainage foil or the wound care kit for use in wound care. Embodiments further relate to a method for caring for a wound.

### BACKGROUND

Drainage foils are required in various types of clinical pictures or traumas, in particular during or after operations in the abdominal cavity, thoracic cavity or pelvic cavity, for example if temporary covering of the open wound and/or drainage of body fluids from the wound region is necessary. Covering of open wounds may be necessary, for example, if several interventions are required daily in order, on the one hand, to allow rapid access to the internal organs and, on the other hand, to reduce a disadvantageous influence of exudate formation in the wound region. By means of temporary covering, a marked drop in mortality can be achieved in some indications. Furthermore, suction, in particular postoperative drainage, of body fluids may be necessary, in order to promote wound healing, since fluid accumulations such as blood or wound secretion in wounds can disrupt the healing process.

Basically, two main requirements are set from a medical point of view for drainage foils suitable for the stated purposes of use. Firstly, good exudate management in the wound region, in particular when used in the open abdominal cavity, thoracic cavity or pelvic cavity, must be achieved, i.e. suction of body fluids in the entire wound region. Furthermore, a reduction in friction between the wound or surrounding organs and the drainage foil should be achieved, while the wound is at the same time sufficiently shielded from the environment. Furthermore, it must be ensured that no contaminants pass through the drainage foil into the open wound or the body of the patient.

EP 0 261 167 B1 describes a liquid-permeable wound covering which is provided for direct contact with the wound base and which has a hydrophobic layer in order to prevent the adherence of the wound covering to the wound region and any contamination of the wound caused thereby. However, satisfactory exudate management in the wound region cannot be obtained with the wound covering described in this document.

To improve exudate management in the wound region, U.S. Pat. No. 7,381,859 B2 proposes a wound covering in which a foam-like layer in which exudates can be absorbed is accommodated between two liquid-permeable, foil-like, web-like elements which can be embodied in the form of plastic films. However, in the case of the wound covering known from this document, it has proven to be problematic that satisfactory suction of exudates cannot take place in edge regions of the wound covering, with the result that complications in wound care occur in these edge regions.

WO 2007/118652 A1 describes a wound spacer grid onto which absorbent secondary dressings can be placed without adhesive and which is equipped with a plurality of three-dimensional perforations to form a first smooth surface and a second surface with a rough handle. When this known wound covering is used, the downstream absorption bodies can be exchanged in order thus to ensure satisfactory wound care over a relatively long period of time. However, even when the wound covering systems described in the said document are used, it has proven to be problematic that satisfactory exudate management cannot be achieved over the entire wound region, in particular in the case of wounds lying deep in the interior of the body.

EP 2 424 477 B1 describes a double-walled foil for wound covering which has an open drainage space. However, in the case of the wound covering known from this document, it has proven to be problematic that satisfactory covering of the wound regions cannot be achieved in the case of complex wound regions, in particular wounds lying deep in the body. Furthermore, the production of the known wound covering has proven to be problematical with regard to the production efficiency.

US 2022/0152287 A1 discloses a wound care arrangement comprising a multilayer drainage foil and a fluid-tight fastening foil.

### SUMMARY

In view of the problems described above, embodiments provide a drainage foil with which good exudate management over the entire wound region, in particular even in the case of complex wounds, for example surgical wounds of the internal organs and/or of the locomotor apparatus, is possible without contamination of the wound. Furthermore, embodiments provide improved methods for producing drainage foils.

### Method for Producing a Double-Walled Drainage Foil

This object is achieved by the claims specified herein and by further development of methods and a tubular drainage foil according to the claims herein.

In the method according to embodiments, by connecting the first web-shaped element and the second web-shaped element and perforating the web-shaped elements, an open drainage space is formed which allows a capillary action between the individual web-shaped elements, with which, on the one hand, body fluids, in particular exudates, are prevented from escaping from the drainage space and, on the other hand, distribution of the body fluids over the entire drainage area is possible, without additional measures, such as providing additional absorption bodies, being absolutely necessary for this purpose. In this way, good exudate management is made possible over the entire wound region, because there are also no edge regions without capillary function which disadvantageously influence the exudate management. The method makes it possible to produce drainage foils which can be cut to any size or any structure of wounds, even for endoluminal application, without problems and furthermore ensure the desired drainage function over the entire wound area. In this case, the method can comprise the drainage foil being configured with a smooth surface which prevents adhesion of tissues and cell wound base or in the case of the surrounding organs. Furthermore, the method can comprise the entry of body fluids into the drainage space of the drainage foil being promoted by the web-shaped elements themselves being formed from a liquid-permeable material.

In this case, the stability of the drainage foils produced by the method herein can be improved if the at least one opening allowing the passage of body fluids is formed by a channel which extends starting from the first or second web-shaped element in the direction of the other web-shaped element and opens into the drainage space, the channel wall of which channel is formed integrally with the respective web-shaped element by perforating the web-shaped element.

In the last-described embodiment, the exudate management can be improved particularly effectively if the opening or the channel is provided in such a way that the cross-sectional area of the opening or of the channel is reduced in a plane extending perpendicularly to a depth direction of the drainage foil starting from the one web-shaped element in the direction of the other web-shaped element, in particular to obtain a capillary action promoting the entry of body fluids into the drainage space. In this way, on the one hand, the removal of exudate from the wound region is assisted and, on the other hand, backflow from the drainage space into the wound region is prevented.

Within the scope of embodiments, it has proven to be particularly favorable with regard to effective exudate management if the at least one opening allowing the passage of body fluids has a diameter in a range from 100 micrometer (μm) to 2000 μm, preferably in a range from 300 μm to 700 μm, more preferably in a range from 400 μm to 600 μm. It has been found that an excellent capillary action can be achieved by at least one, preferably a multiplicity of opening(s) allowing the passage of body fluids and having such a diameter.

Furthermore, it has been found that particularly good exudate management and particularly good stability of the drainage foil are ensured if the first web-shaped element and the second web-shaped element are connected at a multiplicity of fastening regions, preferably at a multiplicity of fastening regions formed in a grid arrangement. In the sense of an excellent suction capacity of the drainage foil, a distance between in each case adjacent fastening regions is preferably 2 millimeters (mm) or more, preferably 3 mm or more, more preferably 5 mm or more. The multiplicity of fastening regions can be arranged, for example, in a rectangular grid. An arrangement in a rectangular grid allows particularly efficient production of the drainage foil.

The web-shaped elements can be connected while simultaneously ensuring the drainage space producing the drainage action via a multiplicity of punctiform fastening regions preferably formed in a grid arrangement. The connection can take place here by means of welding, adhesive bonding or other fixed types of connection. However, the removal of exudates should not be hindered, but rather assisted, by the connection. It has been found that connection by means of ultrasonic welding not only allows more efficient production of the drainage foil than other methods for connecting the web-shaped elements, but also produces particularly stable drainage foils, the structural integrity of which is ensured even under mechanical load, for example in the case of endoluminal application. In particular, it has been found that connection by means of ultrasonic welding allows production of particularly durable drainage foils and a particularly advantageous bond strength of 0.5 Newtons (N)/25.4 mm or more can be achieved.

It has proven favorable if the individual fastening regions have an area of 5 millimeters squared (mm2) or less, in particular 3 mm2 or less, particularly preferably 2 mm2 or less, in a sectional plane extending perpendicularly to the depth direction, wherein the distance between individual fastening regions or individual grid points of the fastening regions formed in a grid arrangement is 2 mm or more, in particular 3 mm or more, particularly preferably 5 mm or more.

Irrespective of whether the fastening takes place by welding, adhesive bonding or other types of connection, a material bridge connecting the inner boundary surfaces of the web-shaped elements to one another can be formed in the fastening regions. It has been found that such a material bridge promoting the stability of the overall structure of the drainage foil can be produced particularly precisely by means of ultrasonic welding. In order to obtain the desired capillary action in the drainage space, the distance between the inner boundary surfaces of the web-shaped elements is 5 mm or less, preferably 4 mm or less, particularly preferably 2 mm or less. In this case, the distribution of the exudates over the entire area of the drainage foil using the capillary action in the drainage space can be ensured if the distance between the inner boundary surfaces of the web-shaped elements is 0.05 mm or more, in particular 0.1 mm or more, particularly preferably 0.3 mm or more. In order to produce high-quality drainage foils, it has proven particularly favorable if the connection of the first web-shaped element and the second web-shaped element is carried out by means of ultrasonic welding, since in this way the desired distance between the first and second web-shaped elements, in particular between an inner boundary surface of the drainage space formed by the first web-shaped element and an inner boundary surface of the drainage space formed by the second web-shaped element, can be produced precisely and in a quality-ensuring manner. It has been found that production of the drainage foil by means of ultrasonic welding allows higher-quality drainage foils than if the distance between the inner boundary surfaces is ensured only by the depth of, in particular, funnel-shaped openings, since perforation typically takes place only with limited accuracy.

For the use of the drainage foil in wound care, it has proven particularly advantageous if the drainage foil has a basis weight in a range from 30 grams per square meter (g/m2) to 90 g/m2, preferably 40 g/m2 to 80 g/m2, more preferably 50 g/m2 to 70 g/m2. The first web-shaped element and/or the second web-shaped element may comprise or be a plastic foil, wherein the plastic foil preferably comprises polyethylene, polypropylene, polyethylene terephthalate, polyurethane, polytetrafluoroethylene, polyhydroxybutyrate, polylactide and/or cellulose. Plastic foils of this type have the advantage that they effectively prevent penetration of pathogens, for example bacteria, and adhesion and ingrowth of tissue. The plastic foil may comprise, in particular, polyethylene, preferably low-density polyethylene (LDPE). It has been found that web-shaped elements or plastic foils comprising polyethylene, in particular low-density polyethylene (LDPE), are particularly advantageous with regard to the production and use of drainage foils. The first web-shaped element and/or the second web-shaped element may comprise polyethylene, polypropylene, polyethylene terephthalate, polyurethane, polytetrafluoroethylene, polyhydroxybutyrate, polylactide and/or cellulose, preferably polyethylene, more preferably low-density polyethylene (LDPE). The structure of the web-shaped elements can be produced by means of a non-woven production process. In this case, synthetic and also natural (e.g., silk, cotton) fibers, and also inorganic fibers (glass ceramic . . . ) or metal (silver fibers) can be used for producing the non-wovens or also tissues. It is furthermore envisaged that the web-shaped elements are equipped with an antibacterial or bacteriostatic layer on one side or on both sides. The antibacterial or bacteriostatic action can be realized, for example, by polyhexamethylene biguanide (PHMB), silver, chlorhexidine, etc. With regard to avoiding the tendency of the drainage foils produced by the method to stick together, at least one web-shaped element may have a hydrophilic or hydrophobic finish. Furthermore, the application of swellable materials is also envisaged.

With regard to the use of the drainage foil produced by the method in wound care, it has proven particularly advantageous if the drainage foil has a tensile strength of 7 N/25.4 mm or more, an elongation at break of 40% or more, a porosity of 75 m3/m2/min or more, and/or a bond strength of 0.5 N/25.4 mm or more. Effective exudate management while simultaneously ensuring the structural integrity of the drainage foil can thus be ensured. The tensile strength is preferably measured in accordance with DIN EN ISO 527. The drainage foil may, for example, have a tensile strength of 7 N/25.4 mm or more, measured in accordance with DIN EN ISO 527. The elongation at break is preferably measured in accordance with DIN EN ISO 527. The drainage foil may, for example, have an elongation at break of 40% or more, measured in accordance with DIN EN ISO 527. The bond strength is preferably measured on the basis of Edana NWSP 401.0.R0. The drainage foil may, for example, have a bond strength of 0.5 N/25.4 mm or more, measured on the basis of Edana NWSP 401.0.R0 or measured in accordance with Edana NWSP 401.0.R0. The porosity is a measure of the ratio of the cavity volume to the total volume of the drainage foil.

In the case of drainage foils produced by the method herein, the at least one opening allowing the passage of body fluids, in particular in relation to a plan view of a planar extent of the first or second web-shaped element, may have a round, circular and/or oval shape. Such a shape allows effective passage of body fluids from the wound region into the open drainage space of the drainage foil.

In the case of the drainage foils produced by the method herein, the desired removal of the exudate can be achieved while ensuring a satisfactory overall stability of the drainage foil if at least one web-shaped element, preferably both web-shaped elements, has/have a multiplicity of openings arranged preferably in a grid shape, in particular arranged in a rectangular grid, wherein the distance between adjacent openings or grid points of a web-shaped element is 15 mm or less, preferably 5 mm or less, in particular 3 mm or less. With regard to the desired overall stability of the drainage foils produced by the method, it has furthermore proven particularly expedient if the mouths of the openings arranged in one of the web-shaped elements are arranged in a projection along the depth direction between the mouths of the openings arranged in the other web-shaped element. In order to prevent collapse of the overall structure of the drainage foil, in particular when applying negative pressure, it has proven particularly expedient if at least one channel forming an opening in a web-shaped element, preferably a multiplicity of such channels, extends in the depth direction over 50% or more of the total depth of the drainage space.

In order to ensure the desired permeability of the web-shaped elements, it has proven expedient if the perforation is carried out in such a way that the mouth area, facing the drainage space, of the individual openings in a plane running perpendicularly to the depth direction is 0.1 mm2 or more, in particular 0.5 mm2 or more, particularly preferably 1 mm2 or more. The desired capillary action can be achieved while avoiding a backflow of fluid from the drainage space into the wound region if the perforation is carried out in such a way that the mouth area of the openings in the drainage foil obtained is 5 mm2 or less, in particular 4 mm2 or less, particularly preferably 3 mm2 or less.

As already explained above, the openings in the channels forming web-shaped elements are formed by perforations in the web-shaped elements. In this connection, in order to obtain a smooth surface with which adhesion of tissues or cells to the wound base or in the case of the surrounding organs can be effectively suppressed, it has proven expedient if the channel wall is of arcuate design at least in sections in a sectional plane extending parallel to the depth direction and merges continuously into the boundary area of the web-shaped element.

The method herein may comprise attaching an adhesive, in particular a glue, an adhesive material and/or a hook-and-loop fastener, in particular one or more barbs, to the drainage foil. Such adhesives, adhesive materials or hook-and-loop fasteners make it possible for the drainage foil produced to be able to be fastened to other structures, for example to body parts, surgical devices, absorption bodies, tubes, wires, stents, insertion aids and/or devices for negative pressure therapy. For example, the method may comprise attaching the adhesive, adhesive material and/or hook-and-loop fastener to two opposite edges of the drainage foil. Such a drainage foil can then be used to wrap around a structure, for example selected from absorption bodies, tubes, wires, stents, insertion aids and devices for negative pressure therapy, and to fix the drainage foil to the structure by connecting the adhesive, adhesive material or hook-and-loop fastener of the two opposite edges.

In terms of production technology, it has proven particularly advantageous if step ii) is carried out before or after step iii), and/or step iv) is carried out after step iii) and before or after step ii).

The method may furthermore comprise forming a three-dimensional, preferably tubular structure from the drainage foil. The advantage of such three-dimensional structures, for example tubular structures, is that they can be used for more complex wounds, for example deep wounds, or in deep regions of the body. In particular, such three-dimensional structures, such as tubular structures, can even be used endoluminally, for example in the gastrointestinal tract or in fistula ducts. The drainage foil can be tubular, for example for an endoluminal application and/or for receiving a drainage tube.

In terms of production technology, it has proven particularly expedient if such a three-dimensional structure is formed by welding, in particular ultrasonic welding or laser welding, by thermal bonding, in particular by means of UV or hot air, using an adhesive and/or by deep-drawing, in particular by deep-drawing, the first and second web-shaped elements connected in step iv).

### Method for Producing a Tubular Drainage Foil

The object(s) herein is/are furthermore achieved by a method for producing a tubular drainage foil, which is characterized in that the method comprises the following steps:
a) providing a drainage foil, preferably a double-walled drainage foil with an open drainage space, more preferably a double-walled drainage foil produced according to the method for producing a double-walled drainage foil;
b) folding down, in particular creasing, the drainage foil onto itself about a fold line, in particular crease line, in such a way that the drainage foil has an overlap region which can be connected to a connecting line; wherein, preferably, the drainage foil has a first edge and a second edge running substantially parallel to the first edge and the drainage foil is folded or creased onto itself along the fold line or crease line, respectively, in such a way that the drainage foil has the overlap region, which can be connected to the connecting line, along the first edge and the second edge;
c) connecting the drainage foil along the overlap region, wherein a connecting line is formed; and
d) obtaining a tubular drainage foil.

Furthermore, the object(s) herein is/are achieved by a method for producing a tubular drainage foil, which is characterized in that the method comprises the following steps:
a) providing a drainage foil, preferably a double-walled drainage foil with an open drainage space, more preferably a double-walled drainage foil produced according to the method for producing a double-walled drainage foil;
b) arranging a first edge of the drainage foil on the elastic material in such a way that an overlap region, which can be connected to a connecting line, is formed between the first edge of the drainage foil and the elastic material;
c) connecting the drainage foil and the elastic material along the overlap region between the first edge of the drainage foil and the elastic material, wherein a connecting line is formed;
d) folding down, in particular creasing, the drainage foil onto the elastic material with respect to a fold line, in particular a crease line, in such a way that an overlap region, which can be connected to a connecting line, is formed between a second edge of the drainage foil, preferably a second edge of the drainage foil running substantially parallel to the first edge, and the elastic material;
e) connecting the drainage foil and the elastic material along the overlap region between the second edge of the drainage foil and the elastic material, wherein a connecting line is formed;
f) obtaining a tubular drainage foil.

The object is also achieved by a method for producing a tubular drainage foil, which is characterized in that the method comprises the following steps:
a) providing a drainage foil, preferably a double-walled drainage foil with an open drainage space, more preferably a double-walled drainage foil produced according to the method for producing a double-walled drainage foil;
b) winding the drainage foil about a winding axis in such a way that the drainage foil has an overlap region, which is formed by the winding about the winding axis and can be connected to a connecting line and in which the drainage foil is present in at least two layers;
c) connecting a first layer and a second layer of the drainage foil present in at least two layers in the overlap region, preferably by an adhesive or a connecting means; wherein, preferably, a connecting line is formed;
d) obtaining a tubular drainage foil.

It has been found that the abovementioned methods for producing a tubular drainage foil each efficiently provide a tubular drainage foil which allows uniform drainage performance even in complex wound regions, for example in the case of wounds lying deep in the body. The following statements relate to all three abovementioned methods for producing a tubular drainage foil. Each of the abovementioned methods for producing a tubular drainage foil may have the following properties.

In terms of production technology, it has proven particularly advantageous if the connecting is carried out by welding, in particular ultrasonic welding or laser welding, by thermal bonding, in particular by means of ultraviolet (UV) or hot air, by an adhesive, in particular a glue or a hook-and-loop fastener, and/or by a connecting means, in particular a clamp. In particular, the connecting of the drainage foil along the overlap region, the connecting of the drainage foil and the elastic material along the overlap region between the first edge of the drainage foil and the elastic material, the connecting of the drainage foil and the elastic material along the overlap region between the second edge of the drainage foil and the elastic material, and the connecting of a first layer and a second layer of the drainage foil present in at least two layers in the overlap region can be carried out by welding, in particular ultrasonic welding or laser welding, by thermal bonding, in particular by means of UV or hot air, by an adhesive, in particular a glue or a hook-and-loop fastener, and/or by a connecting means, in particular a clamp.

A connecting means may be, for example, a clamp, a cord, a thread, a seam, for example an elastic seam or a seam with an elastic material, or an annular application aid. For example, a drainage foil can be wound around a winding axis, in particular wound around a drainage tube, and then fixed as a tubular drainage foil by means of a connecting means, for example a clamp or annular application aid. An overlap region, for example an overlap region between a first and second edge of a double-walled drainage foil, can be connected by means of an elastic seam. An elastic seam increases the expandability of the tubular drainage foil.

The tubular drainage foils produced have particularly advantageous properties for medical use if the connecting is carried out by ultrasonic welding, preferably at a speed in a range from 0.1 to 5.0 meters (m)/minute (min), a power in a range from 50 to 500 watts and/or a pressure in a range from 5 Newtons (N) to 100 N. Such a connection allows a stable connecting line which ensures high tensile strength even under mechanical load.

The connecting in the method for producing a tubular drainage foil, in particular the connecting of the drainage foil along the overlap region, the connecting of the drainage foil and the elastic material along the overlap region between the first edge of the drainage foil and the elastic material, the connecting of the drainage foil and the elastic material along the overlap region between the second edge of the drainage foil and the elastic material, and/or the connecting of a first layer and a second layer of the drainage foil present in at least two layers in the overlap region can comprise arranging the drainage foil in an ultrasonic welding device and welding the drainage foil, in particular by means of a welding die, for example an L-shaped welding die, to form a tubular drainage foil. It has been found that tubular drainage foils with corresponding welding dies can be produced efficiently and in high quality.

It has furthermore been found that the properties of the cut edge are particularly suitable for the functionality of the drainage foil if the connecting is carried out by ultrasonic welding using a cutting wheel with a cutting edge radius of 0.2 mm or less, and/or using a cutting wheel with a grinding angle of 150 or less facing the fold line, in particular crease line, and/or with a grinding angle of 750 or less facing away from the fold line, in particular crease line. It is thus possible to prevent material residues which could impair the functionality of the drainage foil from remaining on the cut edge.

A connecting by means of welding, in particular ultrasonic welding, can be carried out, for example, by heating the material in the overlap region, for example the double-walled drainage foil and/or an elastic material, by the sonotrode caused to vibrate by the generator and thus melting it. In order to produce a weld seam, a cutting wheel can be used, for example a cutting wheel which can weld and cut at the same time. By means of a cutting wheel which can weld and cut at the same time, a weld seam is produced in which the excess material is removed at the same time during the welding process. A conventional cutting wheel can also be used in order to produce a connecting line. Ultrasonic welding can be carried out, for example, with a Nucleus Rotosonic DX1-TC 12 flatbed ultrasonic welding apparatus, for example with an ultrasonic frequency of approximately 35 kilohertz (kHz).

In order to provide a connecting line which is as smooth as possible, in particular in order to prevent any functional impairments of the tubular drainage foil which occur as a result of a rough connecting line, an edge strip of the drainage foil arranged on a side of the connecting line facing away from the fold line, in particular crease line, can be removed during the connecting or after the connecting, in particular by punching. Furthermore, punching can be used in order to remove residual material which is not required.

It has been found that it is particularly advantageous for the functionality of the tubular drainage foil if the method is carried out in such a way that the connecting line, for example weld seam, has a width of 2 mm or less, preferably 1 mm or less, more preferably 0.5 mm or less, along a circumferential direction of the tubular drainage foil. In order to prevent any functional impairment of the product, it is particularly advantageous if a connecting line which is as narrow as possible, in particular weld seam, is provided. Furthermore, it has proven particularly expedient for ensuring the functionality in the entire region of the tubular structure if the connecting line has a thickness of 1 mm or less, preferably 750 μm or less, more preferably 450 μm or less, along a radial direction of the tubular drainage foil or depth direction, in particular extending perpendicularly to the tube axis. Depending on the way in which the connecting is carried out in the method for producing tubular drainage foils, the material can melt in the overlap region, in particular at the connecting line, and thus form a material bridge, for example if the connecting is carried out by means of welding, in particular ultrasonic welding.

According to embodiments, the method for increasing the stability of the drainage foil may comprise attaching a reinforcing element, in particular a reinforcing strip or a reinforcing foil, in a region of the connecting line, preferably by arranging the reinforcing element in the overlap region before the connecting, and/or at at least one tube end. A reinforcing strip can be provided, for example, by the drainage foil being provided in double layers in the region to be reinforced or a reinforcing material, for example a polyethylene foil, being fastened in the region to be reinforced. It has been found that a foil, in particular a polyethylene foil, has a particularly advantageous reinforcing effect as reinforcing element.

For example, the reinforcing element can additionally be co-welded during the connecting by the reinforcing element being arranged in the overlap region before the connecting, in order thus to increase the tear strength of the drainage foil, in particular the tear strength at the connecting line. The reinforcing element not only has the advantage that it increases the tear strength of the drainage foil, but can also offer further advantages, for example be embodied as an X-ray-contrast-capable reinforcing element. This allows, for example, precise placement of the tubular drainage foil on the target structure under the control of X-ray imaging.

The excellent structural integrity of the tubular drainage foil can furthermore also be further increased if the tubular drainage foil is formed at at least one tube end in a circumferential direction with a reinforcing region in which the drainage foil is present in at least two layers. This is advantageous above all for those applications in which aids, for example an absorption body, tube, wire, stent or an insertion aid, are to be inserted into the tubular drainage foil, for example during surgical operations or during the production of wound care kits. The tube opening is further stabilized by the reinforcing region, such that any desired aids can be inserted into the tubular drainage foil without there being a risk of tearing of the drainage foil. In terms of production technology, it has proven advantageous if the reinforcing region is formed by folding down a portion of the tubular drainage foil at the tube end, in particular an edge region of the tubular drainage foil, wherein the folding down is carried out in particular in such a way that a tubular element forming the inner boundary surface of the tubular drainage foil forms the outer boundary surface of the tubular drainage foil in the reinforcing region. The method may furthermore comprise fixing, for example punctiform fixing, of the folded-down portion of the tubular drainage foil, in particular by welding, thermal bonding, an adhesive, and/or a connecting means. “Folding down” is to be understood as meaning any shaping, arrangement, bending, winding, or folding of a drainage foil in which an overlap region, in particular an overlap region which can be connected to a connecting line, and/or a reinforcing region with the drainage foil can be achieved.

The methods for producing a tubular drainage foil may comprise rolling up at least one portion of the tubular drainage foil starting from a tube opening at a tube end along a longitudinal tube axis, in particular along a longitudinal tube axis starting from an inner boundary surface in the direction of an outer boundary surface. The tubular drainage foil can be rolled up in portions or completely. It has been found that an application of a rolled-up tubular drainage foil is simplified compared to non-rolled-up drainage foils, since the user can effortlessly roll the tubular drainage foil over structures, for example drainage tubes or probes, in order to sheath them with the tubular drainage foil.

In the sense of a smooth surface of the connecting line on the outer side of the tubular drainage foil, it has proven to be advantageous if, after the connecting, a side of the connecting line facing the fold line, in particular crease line, is rotated completely outwards by inverting the tubular drainage foil. A smooth surface of the connecting line on the outer side ensures high functionality of the tubular drainage foil in the region of the connecting line.

In order to achieve particularly good drainage effects with the tubular drainage foil produced, the drainage foil provided in step a) of the method for producing a tubular drainage foil may be a drainage foil produced according to a method for producing a double-walled drainage foil. The construction of the drainage foil produced prevents collapse of the open drainage space between the two layers, assisted by the fastening regions. Thus, suction distribution over the entire area as far as the edge regions of the tubular drainage foil is ensured and the drainage foil effectively removes the exudate.

### Tubular Drainage Foil

The object of embodiments herein is furthermore achieved by a tubular drainage foil with a first tubular element forming an outer boundary surface and a second tubular element forming an inner boundary surface facing away from the outer boundary surface and the second tubular element being connected to the first tubular element by at least one fastening region, wherein the first tubular element and the second tubular element each comprise at least one opening allowing the passage of body fluids and an open drainage space is formed between the outer boundary surface and the inner boundary surface. In the tubular drainage foil according to embodiments, a capillary action is achieved between the two tubular elements by the open drainage space, which capillary action allows body fluids, in particular exudates, to flow off or be sucked off over the entire tubular structure. An effective exudate management of even more complex wounds, for example wounds lying deep in the interior of the body, in particular even in the case of an endoluminal application, can be ensured by a tubular drainage foil. The tubular drainage foil according to embodiments has the advantage that it can be used not only on the surface of wounds, but can also be arranged between organ structures in order to effectively absorb body fluid, for example exudates, there.

The first tubular element and/or the second tubular element may comprise or be a plastic foil, wherein the plastic foil preferably comprises polyethylene, polypropylene, polyethylene terephthalate, polyurethane, polytetrafluoroethylene, polyhydroxybutyrate, polylactide and/or cellulose. Plastic foils of this type have the advantage that they effectively prevent penetration of pathogens, for example bacteria, and adhesion and ingrowth of tissue. The plastic foil may comprise, in particular, polyethylene, preferably low-density polyethylene (LDPE). It has been found that tubular elements or plastic foils comprising polyethylene, in particular low-density polyethylene (LDPE), are particularly advantageous for the production and use of tubular drainage foils. The first tubular element and/or the second tubular element may comprise polyethylene, polypropylene, polyethylene terephthalate, polyurethane, polytetrafluoroethylene, polyhydroxybutyrate, polylactide and/or cellulose, preferably polyethylene, more preferably low-density polyethylene (LDPE). It is furthermore envisaged that the tubular elements are equipped with an antibacterial or bacteriostatic layer on one side or on both sides. The antibacterial or bacteriostatic action can be realized, for example, by PHMB, silver, chlorhexidine, etc. With regard to avoiding the tendency of the drainage foils produced by the method to stick together, at least one tubular element may have a hydrophilic or hydrophobic finish. Furthermore, the application of swellable materials is also envisaged.

In the sense of the structural integrity of the tubular drainage foil, it has proven favorable if the first tubular element and the second tubular element are connected at a multiplicity of fastening regions, preferably at a multiplicity of fastening regions formed in a grid arrangement. A distance between adjacent fastening regions is preferably 2 mm or more, preferably 3 mm or more, more preferably 5 mm or more, in order to ensure an excellent capillary action of the open drainage space formed between the first tubular element and the second tubular element. The multiplicity of fastening regions can be arranged, for example, in a rectangular grid.

Furthermore, it has proven expedient with regard to an effective capillary action if the individual fastening regions have an area of 5 mm2 or less, in particular 3 mm2 or less, particularly preferably 2 mm2 or less, in a sectional plane extending perpendicularly to a radial direction or depth direction. The depth direction is in particular a direction extending perpendicularly to the boundary surfaces. The radial direction extends in particular perpendicularly to the longitudinal tube axis.

Irrespective of whether the fastening at the fastening region takes place by welding, for example ultrasonic welding, adhesive bonding or other types of connection, a material bridge connecting the tubular elements to one another can be formed in the fastening regions. It has been found that such a material bridge promoting the stability of the overall structure of the tubular drainage foil can be produced particularly precisely by means of ultrasonic welding. In order to obtain the desired capillary action in the drainage space, the distance between the inner boundary surfaces of the tubular elements which delimit the open drainage space is 5 mm or less, preferably 4 mm or less, particularly preferably 2 mm or less. In this case, the distribution of the exudates over the entire area of the tubular drainage foil using the capillary action in the drainage space can be ensured if the distance between the inner boundary surfaces of the tubular elements which delimit the drainage space is 0.05 mm or more, in particular 0.1 mm or more, particularly preferably 0.3 mm or more. The tubular drainage foil has such a distance between the inner boundary surfaces of the tubular elements which delimit the drainage space, preferably in at least 50% of the area of the drainage foil, preferably at least 70% of the area of the drainage foil.

The tubular drainage foil according to embodiments can have a connecting line, preferably a weld seam, which connects the first tubular element and the second tubular element, and/or the first and/or second tubular element and an elastic material. A connecting line can be provided, for example, by welding, in particular ultrasonic welding or laser welding, by thermal bonding, in particular by means of UV or hot air, by an adhesive, in particular a glue or a hook-and-loop fastener, and/or by a connecting means, in particular a clamp. For example, a connecting line can be produced by a first edge of a drainage foil being arranged on a second edge of the drainage foil and the latter being connected to one another, for example by ultrasonic welding, as a result of which a connecting line is produced between the first edge and the second edge, for example a weld seam.

It has been found that it is particularly advantageous for the functionality of the tubular drainage foil if the connecting line, for example weld seam, has a width of 2 mm or less, preferably 1 mm or less, more preferably 0.5 mm or less, along a circumferential direction of the tubular drainage foil. Furthermore, it has proven particularly expedient for ensuring the functionality in the entire region of the tubular structure if the connecting line has a thickness of 1 mm or less, preferably 750 μm or less, more preferably 450 μm or less, along a radial direction of the tubular drainage foil or depth direction.

For the use of the tubular drainage foil in wound care, it has proven particularly advantageous if the drainage foil has a basis weight in a range from 30 g/m2 to 90 g/m2, preferably 40 g/m2 to 80 g/m2, more preferably 50 g/m2 to 70 g/m2. Effective exudate management while simultaneously ensuring the structural integrity of the drainage foil can be ensured if the drainage foil has a tensile strength of 7 N/25.4 mm or more, an elongation at break of 40% or more, a porosity of 75 m3/m2/min or more, and/or a bond strength of 0.5 N/25.4 mm or more. The tensile strength is preferably measured in accordance with DIN EN ISO 527. The tubular drainage foil may, for example, have a tensile strength of 7 N/25.4 mm or more, measured in accordance with DIN EN ISO 527. The elongation at break is preferably measured in accordance with DIN EN ISO 527. The tubular drainage foil may, for example, have an elongation at break of 40% or more, measured in accordance with DIN EN ISO 527. The bond strength is preferably measured on the basis of Edana NWSP 401.0.R0. The tubular drainage foil may, for example, have a bond strength of 0.5 N/25.4 mm or more, measured on the basis of Edana NWSP 401.0.R0 or measured in accordance with Edana NWSP 401.0.R0. The porosity is a measure of the ratio of the cavity volume to the total volume of the drainage foil.

The tubular drainage foil may have a tube opening at at least one tube end. For example, a tubular drainage foil can be provided which has a tube opening at one tube end and has material continuously at the other end of the tubular structure, for example web-shaped elements connected by means of a connecting line and/or tubular elements which form the tube end. Alternatively, a tubular drainage foil can be provided which has a tube opening at both tube ends. For some applications, for example for drainage in the case of wounds lying deep in the body, it has proven to be particularly advantageous if the tubular drainage foil has a tube opening at one tube end and no tube opening at another tube end.

The tubular drainage foil may have a reinforcing element, in particular a reinforcing strip or a reinforcing foil, at at least one tube end and/or along the connecting line. A reinforcing strip may be, for example, a region in which a material, for example a double-walled drainage foil, in particular a drainage foil produced according to the method for producing a double-walled drainage foil, is present in two layers, and/or a region in which a reinforcing material, for example a polyethylene foil, is fastened to the first and/or the second tubular element. It has been found that a foil, in particular a polyethylene foil, has a particularly advantageous reinforcing effect as reinforcing element. The structural integrity of the tubular drainage foil is particularly promoted if the connecting line and/or the at least one tube opening has/have a reinforcing element.

The excellent structural integrity of the tubular drainage foil is furthermore further increased if the tubular drainage foil has a reinforcing region at at least one tube end in which the drainage foil is present in at least two layers. This is advantageous above all for those applications in which aids, for example an absorption body, tube, wire, stent or an insertion aid, are to be inserted into the tubular drainage foil. The tube opening is further stabilized by the reinforcing region, such that any desired aids can be inserted into the tubular drainage foil without there being a risk of tearing of the drainage foil. In terms of production technology, it has proven advantageous if the reinforcing region is formed in the form of a two-layer edge which is formed by folding down a portion of the tubular drainage foil at the tube end, wherein the folding down is carried out in particular in such a way that the second tubular element forming the inner boundary surface forms the outer boundary surface of the tubular drainage foil, in particular the outer boundary surface of the reinforcing region of the tubular drainage foil, in the reinforcing region. The folded-down edge can be fixed in a punctiform manner.

The outer boundary surface of the tubular drainage foil, the inner boundary surface of the tubular drainage foil and/or an inner lumen of the tubular drainage foil comprised by the inner boundary surface of the tubular drainage foil may comprise/comprise a sliding aid and/or an insertion aid. The ease of use and the simplicity of the application can be further increased by such sliding aids and/or insertion aids. For example, an outer boundary surface and/or an inner boundary surface of the tubular drainage foil can be configured with a low-friction surface, in particular with an anti-stick coating and/or a microrough surface, in order to facilitate insertion. In particular, the outer boundary surface and/or the inner boundary surface of the tubular drainage foil may comprise or be coated with silicone, polytetrafluoroethylene (PTFE) and/or moisture in order to facilitate insertion. Sliding aids which markedly increase the sliding capability are, for example, a water-based lubricant, a glycerol-based lubricant, a polymeric gel, in particular an anti-allergenic polymeric gel, an endoscopic lubricant, in particular an endoscopic sliding gel, and/or an aqueous solution, in particular a sodium chloride solution. It is particularly advantageous if the substances used as sliding aids can dissolve without residue, in particular in an aqueous solution, in order to prevent substance residues from remaining on the product. Insertion aids which comprise a textile and/or a plastic are particularly suitable as insertion aids. The insertion aids may comprise a low-friction surface, in particular an anti-stick coating and/or a microrough surface. For example, the insertion aid may comprise a textile and/or a plastic, wherein the textile or the plastic comprises or is coated with silicone, polytetrafluoroethylene (PTFE) and/or moisture. The insertion aid may be a tubular insertion aid arranged in the inner lumen of the tubular drainage foil, for example a tubular insertion aid which has a tube opening or two tube openings. In particular, the insertion aid may have a tube opening at one tube end and a closed tube end at the other tube end. A tubular insertion aid can be configured with a predetermined breaking point at a closed tube end of the tubular insertion aid. The tubular drainage foil can be provided in a single-lumen or multi-lumen manner.

So that the tubular drainage foil is particularly advantageous for a multiplicity of applications, for example an endoluminal application, an application in gynecology or urology, and an application in thoracic, visceral and/or hip surgery, the tubular drainage foil can have a length in a range from 0.5 cm to 40 cm, preferably 2 cm to 30 cm, more preferably 5 cm to 20 cm. However, longer or shorter tubular drainage foils can also be provided. For example, tubular drainage foils with a length of 10 cm or longer, or 20 cm or longer, are suitable for intraluminal applications in the gastrointestinal tract and for fistula ducts. In the case of an application in various wound cavities, the length can be adapted to the wound cavity; for example, tubular drainage foils with a length of 10 cm or less can be provided for wound cavities, or for smaller wound cavities with lengths of 5 cm or less, for example 2 cm or less.

The tubular drainage foil is furthermore particularly suitable for a multiplicity of applications in the sense of effective wound management if the tubular drainage foil has an outer diameter in a range from 1 mm to 60 mm, preferably 2 mm to 50 mm, more preferably 4 mm to 30 mm. However, tubular drainage foils with a larger or smaller outer diameter can also be provided. For applications in the case of narrow wounds and/or in deep regions of the body, for example on the pancreas, applications in gynecology or urology, and in the case of endoluminal applications, a tubular drainage foil with a small outer diameter is particularly suitable, for example tubular drainage foils with an outer diameter of 5 mm or less, in particular 4 mm or less. The tubular drainage foil has the advantage that even in the case of a small outer diameter of the drainage foil, uniform drainage performance is ensured and clogging of the drainage outflow by tissue is prevented. The tubular drainage foil thus enables active drainage of liquids even in the case of a very small outer diameter. In the case of invasive surgery, for example on the hip, in particular in the case of large wound regions, tubular drainage foils with a larger outer diameter can also be used, for example with an outer diameter of 6 mm or more. According to the accessibility and size of the wound region, a tubular drainage foil with a suitable length and a suitable outer diameter can be provided.

### Wound Care Kit

The object of embodiments herein is furthermore achieved by a wound care kit, which comprises a tubular drainage foil produced according to the method for producing a tubular drainage foil or the tubular drainage foil, and at least one further constituent, for example selected from an absorption body, a tube, a wire, a stent and an insertion aid. In the sense of particularly simple handling of the wound care kit, it has proven to be advantageous if at least one portion of the at least one further constituent is arranged in an inner lumen of the tubular drainage foil comprised by an inner boundary surface. For particularly efficient negative pressure therapy, the wound care kit may comprise, for example, the tubular drainage foil and a drainage tube, wherein a portion of the drainage tube is arranged in an inner lumen of the tubular drainage foil. The drainage tube may be connected to a negative pressure source. The at least one further constituent may be arranged in portions or completely in the tubular drainage foil.

In a particularly preferred embodiment, the absorption body is a sponge, foam, in particular polyurethane or polyvinyl alcohol foam, or gauze. It has been found that a wound care kit with such an absorption body allows efficient suction of liquids from body regions, in particular wound regions. Adhesion of tissue to the absorption body is prevented if the absorption body is arranged in the inner lumen of the tubular drainage foil comprised by an inner boundary surface. Liquids can thus be suctioned efficiently and atraumatically from wound regions.

In the sense of particularly advantageous suction efficiency, it has proven to be favorable if a tube comprised by a wound care kit is a drainage tube. The drainage tube can be single-lumen or multi-lumen. A wound care kit with a drainage tube allows continuous suction of liquids from the wound region, and furthermore allows suction of large liquid volumes. Since the tubular drainage foil can be provided in any dimensioning, in particular with any outer diameter, any length and any shape, the wound care kit can even be provided with tubes and/or be used with tubes which have a complex geometry, for example a multi-lumen geometry.

The wound care kit can have a length in a range from 0.5 cm to 250 cm, preferably 10 cm to 200 cm. The length of the wound care kit can be optimized for the respective application; for example, a wound care kit with a length of at least 10 cm or at least 20 cm can be provided for intraluminal applications in the gastrointestinal tract or for an application in fistula ducts. For an application in wound cavities, the length can be adapted to the respective wound cavity; for example, the wound care kit can have a length of less than 10 cm, less than 5 cm, or less than 2 cm, depending on the size or depth of the wound cavity.

### Medical Use and Method for Caring for a Wound

Embodiments furthermore relate to the tubular drainage foil for use in wound care and the wound care kit for use in wound care, for example for draining a wound and/or for use in a method for caring for a wound. The tubular drainage foil or the wound care kit are preferably used for drainage, for example during surgical operations and/or postoperatively. The tubular drainage foil or the wound care kit is preferably used for draining body fluids from wound regions, for example during operations. The tubular drainage foil and the wound care kit allow simple and reliable suction of liquids from a wound. The tubular drainage foil and the wound care kit can be used, for example, endoluminally, for example in the gastrointestinal tract, in gynecology and urology, in surgical operations, for example in thoracic, visceral and hip surgery, and in negative pressure therapy. The tubular drainage foil or the wound care kit can be used for use in drainage, in particular within the scope of a surgery. Thus, blood, wound secretions and/or tissue fluid, the accumulation of which in the wound cavity can be prevented by applying the drainage, usually accumulate during a surgical intervention. In this way, the healing process can be markedly facilitated.

The tubular drainage foil or the wound care kit can be used with particular advantage in combination with negative pressure therapy for wound care. In this case, the tubular drainage foil or the wound care kit is used as a direct interface between the wound, in particular the wound base, and a wound filler or a region of exudate removal, for example a drainage tube. With particular advantage, the tubular drainage foil or the wound care kit can be used for use in wounds on the internal organs. In this case, the tubular drainage foil is applied to the wound, wherein the tubular drainage foil offers a drainage function and a very smooth surface in order to prevent adhesion or friction of tissue and foil. The tubular drainage foil or the wound care kit can be anatomically preformed, but can also be configured such that it can be tailored to specific circumstances. When used in combination with negative pressure therapy, a filling medium such as gauze or foam can be filled into the tubular drainage foil or the wound care kit and/or a drainage tube can be inserted. The tube used for discharging the exudates or for wound drainage can be led out of the interior of the body, for example the abdominal cavity, and connected to a negative pressure source, which can be ensured by a pump or in the hospital by a central home care. With the negative pressure source, the exudate can then be removed from the wound by means of the tubular drainage foil or the wound care kit, as a result of which good exudate management is achieved.

In the case of medical use of the tubular drainage foil or the wound care kit, the following advantages result:
The openings allowing the passage of body fluids assist the removal of exudate and prevent backflow of the exudate into the wound space (capillary action).
The tubular drainage foil prevents adhesion of tissues or cells to the wound base or in the case of the surrounding organs.
The tubular drainage foil or the wound care kit can be tailored to any size or structure of the wound region without impairment of the drainage function and thus also makes it possible to care for complex wounds.

Embodiments are also directed to a method for caring for a wound, which is characterized in that a tubular drainage foil or a wound care kit is contacted with a wound of a patient. In particular, the tubular drainage foil or the wound care kit is contacted with the wound of a patient in such a way that body fluids, for example exudate or blood, can flow off from the wound into the tubular drainage foil or into the wound care kit. The tubular drainage foil and the wound care kit allow effective flow off or suction of body fluids, in particular blood or wound fluid, from wounds. The method for caring for a wound has the advantage that body fluids are effectively suctioned and penetration of bacteria or other pathogens into the wound region is prevented.

The method for caring for a wound may be a method for draining a wound. The method for caring for a wound can be used in all fields of medicine and in all regions of the body, for example endoluminal applications, applications in gynecology or urology, and applications in thoracic, visceral and/or hip surgery are included. As explained above, a length and/or an outer diameter of the tubular drainage foil or of the wound care kit can be adapted to the requirements of the respective wound regions, for example small outer diameters can be used for an endoluminal application in the gastrointestinal tract. The method for caring for a wound can be used in any region of the body and for any wound, since the shape of the tubular drainage or of the wound care kit can be adapted according to the shape of the respective region of the body, of the wound or of the required drainage region. It is provided to provide tubular drainage foils or wound care kits with any shape, any size and any outer diameter which are suitable for any wound or any medical use. For example, the tubular drainage foils can be round or cylindrical, for example for an application in the gastrointestinal tract or in fistulae, or be configured as flat tubular drainage foils, in particular flat drainage foils. Furthermore, the tubular drainage foil can be provided in any dimensioning.

A method for caring for a wound has the advantage over other methods for caring for wounds that the tubular drainage foil adheres less to tissue, for example in comparison with absorption bodies such as polyurethane foam, as a result of which the risk with regard to injuries and bleeding is reduced. The method is therefore even suitable for sensitive body sites, for example in the region of blood vessels or pre-damaged tissue, and for patients with an increased risk of bleeding. Furthermore, it can be used in a more versatile manner since a tubular drainage foil or a wound care kit can be used which corresponds to the anatomical requirements and the drainage requirements in the wound region.

### BRIEF DESCRIPTION OF THE FIGURES

Embodiments are explained below with reference to the drawing, to which explicit reference is made with regard to all details which are essential to embodiments and are not highlighted in any more detail in the description. In the drawing shows:

FIG. 1 a schematic illustration of an ultrasonic welding apparatus.

FIG. 2 a schematic illustration of a punching apparatus.

FIG. 3 a schematic illustration of an apparatus for inverting the tubular drainage foil.

FIG. 4 a schematic illustration of a creasing technique or winding technique for folding down, in particular creasing, the drainage foil about a fold line, in particular crease line, or for winding the drainage foil about a winding axis in such a way that the drainage foil has an overlap region which can be connected to a connecting line.

FIG. 5 a schematic illustration of a self-adhesive drainage foil which can be used as a tubular drainage foil by winding or folding.

FIG. 6 a schematic illustration of a self-adhesive drainage foil which can be used as a tubular drainage foil by winding or folding, for example about a drainage tube.

FIG. 7 a schematic illustration of a connecting line, in particular a weld seam.

FIG. 8 a schematic illustration of attaching a reinforcing element to the drainage foil.

FIG. 9 a schematic illustration of an insertion aid.

FIG. 10 a schematic illustration of rolling up the tubular drainage foil and a rolled-up tubular drainage foil.

FIG. 11 a schematic illustration of a tubular drainage foil with elastic material.

FIG. 12 a schematic illustration of a tubular drainage foil with edge reinforced by folding down.

FIG. 13 schematic illustrations of tubular drainage foils.

FIG. 14 a schematic illustration of a tubular drainage foil with tube.

FIG. 15 a schematic illustration of a tubular drainage foil with tube and absorption body.

FIG. 16 a schematic illustration of a wound tubular drainage foil with tube.

### DETAILED DESCRIPTION

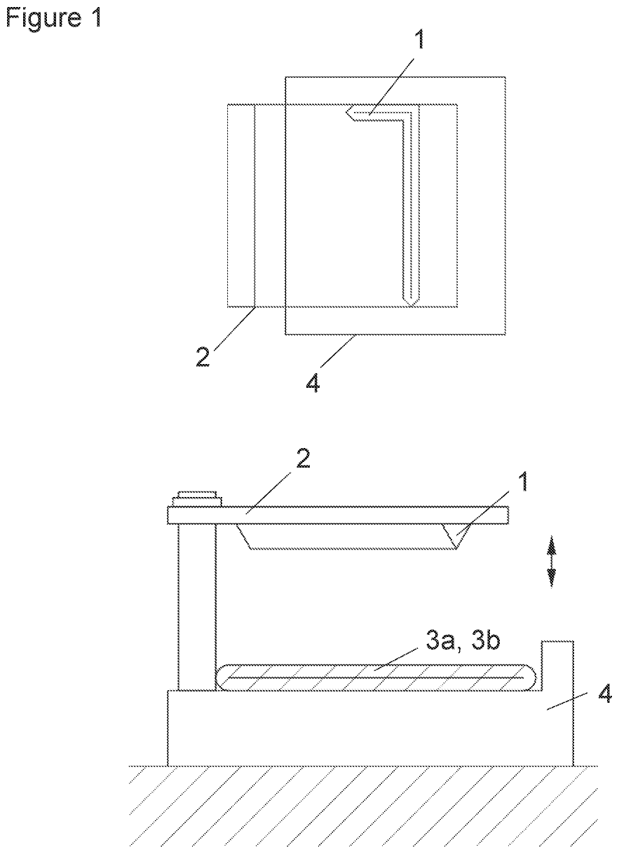

The ultrasonic welding apparatus illustrated in FIG. 1 can be used for producing the tubular drainage foil 3a. For example, the ultrasonic welding apparatus can be used in order to produce a tubular drainage foil 3a by simultaneous punching and welding; here, as a result of the ultrasonic welding, the overlapping material is connected in the overlap region to form a connecting line 11 and at the same time an edge strip of residual material is removed. The double-walled drainage foil 3b is inserted into the foundation 4 in a folded-down manner and fixed. The ultrasonic welding die 1, for example an L-shaped ultrasonic welding die 1, is located in the bracket 2 positioned above the foundation 4. A generator induces the required voltage into the welding die 1. The welding die 1 moves downwards and welds the contour of the tubular drainage foil 3a. After the welding operation has ended, the bracket 2 moves back into the starting position. The tubular drainage foil 3a can then be removed. The left-hand illustration shows the plan view of the ultrasonic welding apparatus and the right-hand illustration shows a side view.

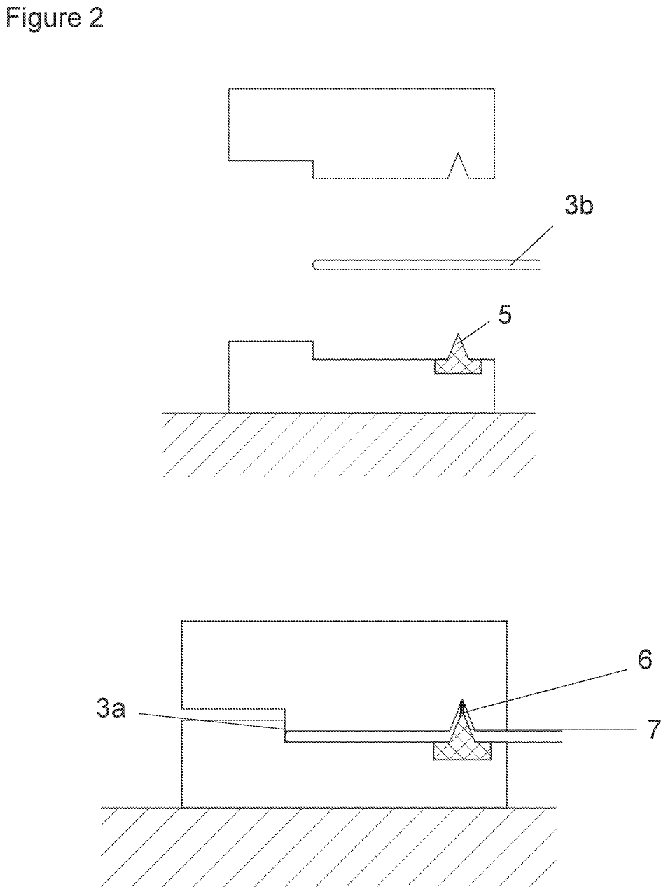

The punching apparatus illustrated in FIG. 2 can be used for producing the drainage foil 3. A double-walled drainage foil 3b provided is inserted into the lower die half of the punching tool in a folded-down manner on one side. A cutting tool with integrated heating is located in the lower die half of the punching tool. The punching operation is started by moving down the upper die half. In the same operation, the hot and sharp cutting element 5 welds the drainage foil 3b to the cutting edge and separates it, as a result of which a tubular drainage foil 3a with a weld seam 6 is obtained. The foil residue 7 is removed by the punching. The die is then opened by moving up the upper die half and the tubular drainage foil 3a can be removed.

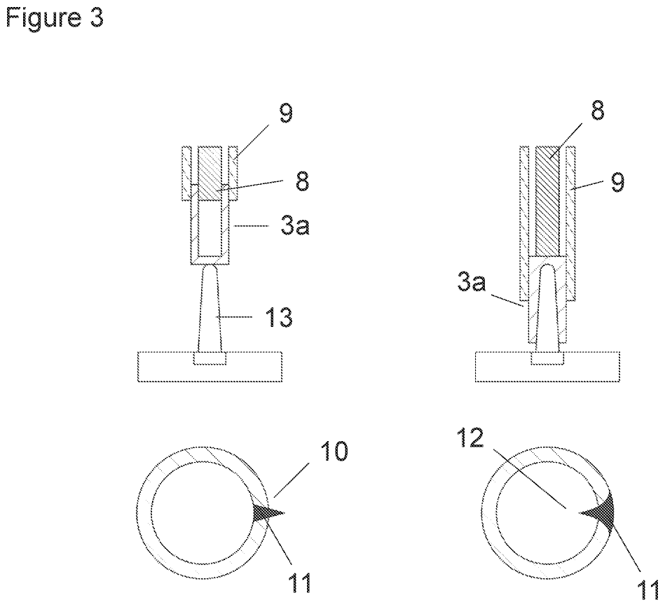

The apparatus illustrated in FIG. 3 can be used for inverting the tubular drainage foil 3a. Firstly, the tubular drainage foil 3a is produced according to one of the methods mentioned, for example by welding the overlap region to form a connecting line 11. For the use in the wound region, in particular for preventing adhesion of tissue, it has proven particularly advantageous if the connecting line 11 has a smooth surface on the outer side of the tubular drainage foil 3a. In order to produce a smooth surface on the weld seam 6, the tubular drainage foil 3a can be inverted from the inside outwards in such a way that the side 10 of the connecting line 11 previously facing away from the fold line, in particular crease line, lies on the side 12 of the connecting line 11 facing the fold line, in particular crease line, after the inverting. The tubular drainage foil 3a can be pulled over the upper movable ram 8 of the illustrated apparatus for turning over or inverting. A sleeve 9 additionally moves over the movable ram 8 and rotates the tubular drainage foil 3a with the aid of the rigid counter ram 13.

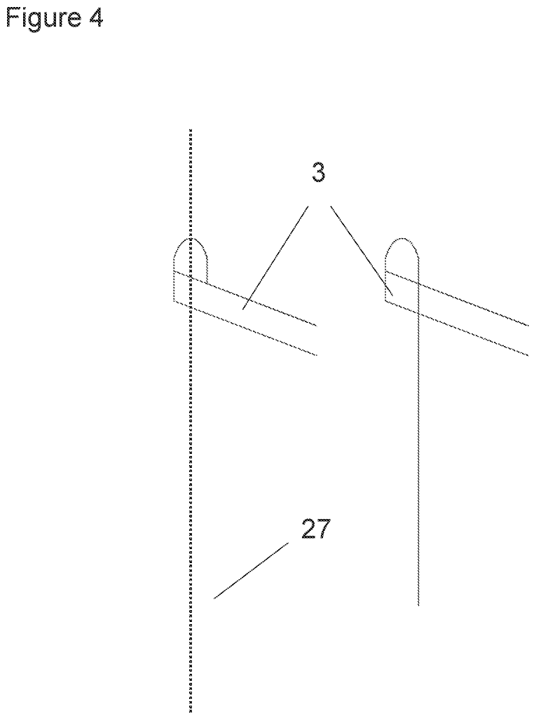

The folding-over or winding technique illustrated in FIG. 4 can be used to place a drainage foil 3 planarly about a winding axis 27, for example over the tip of a tube 16, or to fold it over a tube 16. For example, the drainage foil 3 can be wound about a winding axis 27 in such a way that the drainage foil 3 has an overlap region, which is formed by the winding about the winding axis 27 and can be connected to a connecting line 11 and in which the drainage foil 3 is present in at least two layers. The overlap region can then be connected or fixed in order to obtain a tubular drainage foil 3a, for example by an adhesive 14 or a connecting means, such as a clamp, seam or an annular application aid.

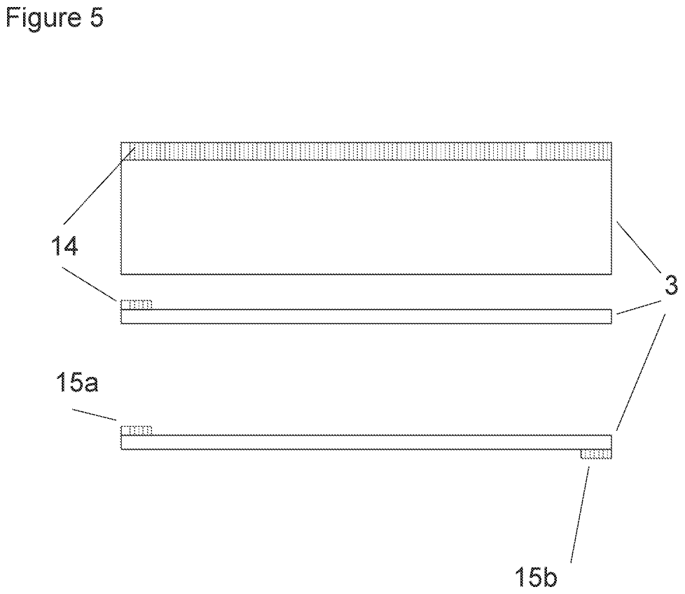

The self-adhesive drainage foil 3 illustrated in FIG. 5 can be fastened to any desired structures, for example to body parts, surgical devices, absorption bodies, tubes, wires, stents, insertion aids 22 and/or devices for negative pressure therapy. An adhesive 14 attached to the drainage foil 3, in particular a glue, an adhesive material and/or a hook-and-loop fastener 15, such as one or more barbs, can be used to enable the drainage foil 3 to be wound or folded around other structures and then fastened in particular in a tubular manner to the structure or around the structure. Such a drainage foil 3 can be used as a self-adhesive tubular drainage foil 3a and has the advantage that it can be adapted in situ to the structural requirements, for example to the size of the wound cavity. During the winding or folding of the drainage foil 3 around the corresponding structure, for example around a drainage tube, the adhesive or adhesive portion of the drainage foil 3 can be fastened to another portion of the drainage foil 3. If the drainage foil 3 fixed in this way to a structure has to withstand particularly high mechanical loads, the drainage foil 3 can additionally be fixed if necessary by means of a seam. The drainage foil 3 can be provided with one or more adhesive portions, in particular the adhesive 14 can be attached to one portion, or the adhesive 14, for example the two complementary constituents of a hook-and-loop fastener 15a, 15b, can be attached to two or more portions which can thereby be attached to one another.

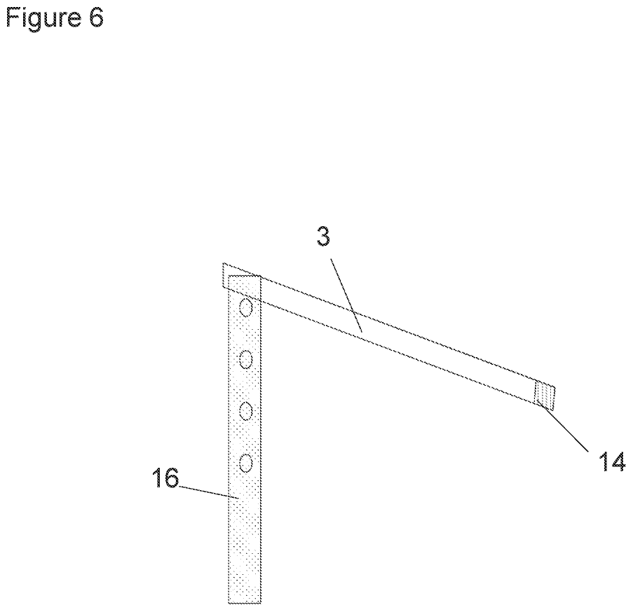

FIG. 6 shows a self-adhesive drainage foil 3 with an adhesive 14 which can be wound around a tube 16 or a probe and can thus be used as a tubular drainage foil 3a.

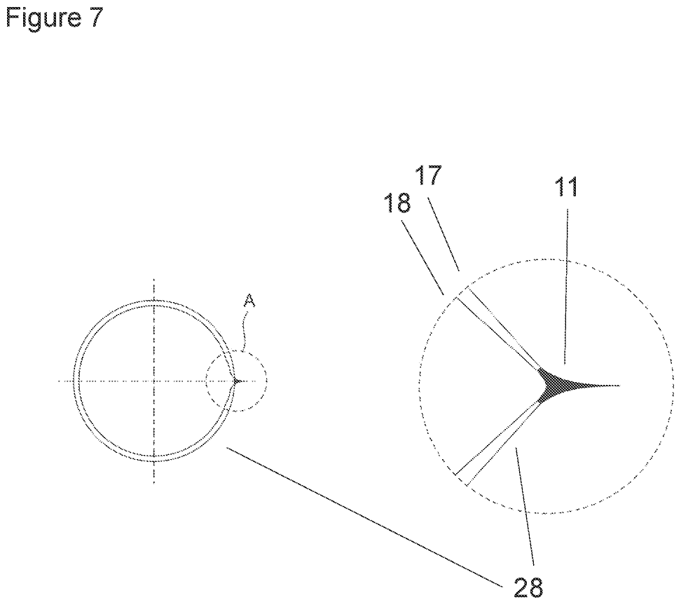

FIG. 7 shows a schematic illustration of a connecting line 11, in particular a weld seam 6, between a first element 17 and a second element 18, for example a first and second web-shaped element or a first and second tubular element. Detail A shows a tapering of the foil thickness by the connection, in particular by a welding operation. The foil is compressed in the region of the connecting line 11, in particular weld seam 6. By compressing the double-walled drainage foil 3b in the region of the connecting line 11 and the cutting wheel angle, a geometry-dependent angle α is produced between the ridge of the connecting line 11 and the outer boundary surface 28 of the tubular drainage foil 3a.

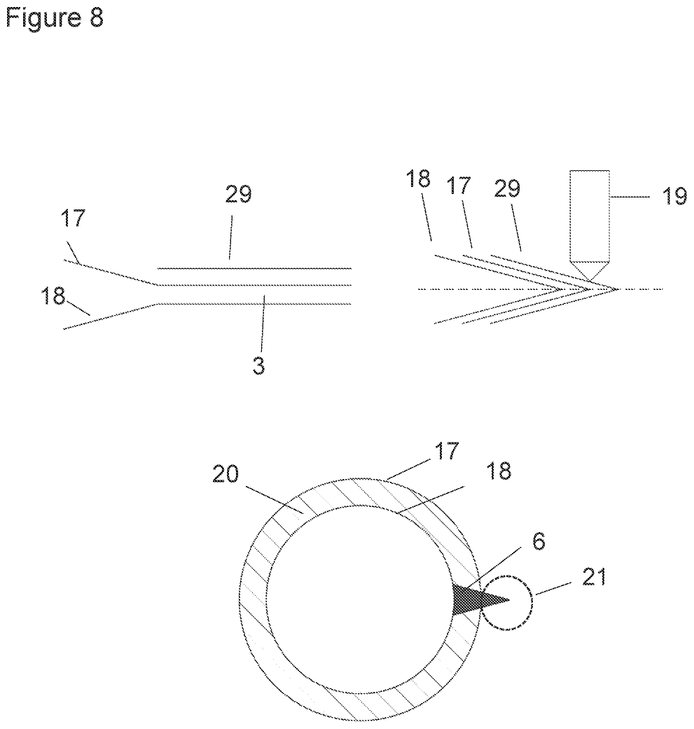

A connecting line 11, for example a weld seam 6, of a perforated drainage foil 3 can represent a critical point of the product in the sense of the foil stability. In order to equip the tubular drainage foil 3a with an open drainage space 20 with particularly excellent strength properties, in particular to improve a strength of a connecting line 11 such as a weld seam 6, it can be reinforced with additional material. FIG. 8 shows a schematic illustration of such an attachment of a reinforcing element 29 to the drainage foil 3 comprising the first element 17 and the second element 18, for example by welding to a cutting wheel 19. An in particular non-perforated polyethylene foil can be used as additional material or as reinforcing element 29. In order to be able to weld the additional foil layer, for example additional in particular non-perforated polyethylene foil, to the drainage foil 3 particularly effectively, the foil layer may be made of polyethylene. As a result of the additionally welded in particular non-perforated polyethylene foil, more material flows into the holes of the drainage foil 3 in the region of the connecting line 11, for example during welding, and stabilizes said holes, as a result of which an excellent strength is achieved. Such a reinforcing element 29, for example a polyethylene foil, markedly increases the tensile load of the weld seam 6. In order to attach the reinforcing element 29, the reinforcing element, for example an additional polyethylene foil, can be placed over the drainage foil 3 and then folded down. During the connecting, for example by ultrasonic welding, the reinforcing element 29 connects to the drainage foil 3 in the overlap region, as a result of which a connecting line 11, in particular a stable weld seam 6, is produced. The residual material 21 of the reinforcing element 29, for example a residual polyethylene foil, can then be removed in the region of the connecting line 11, in particular in the region of the weld seam 6, for example manually or mechanically. The reinforcing element 29, for example a polyethylene foil, can be provided such that it is present only in the connecting line 11, in particular a weld seam 6.

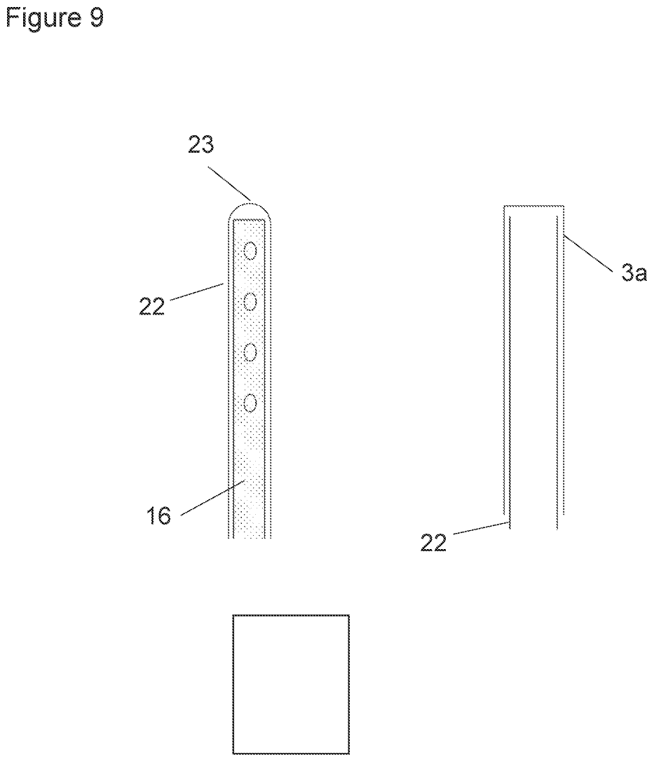

The tubular drainage foil 3a with insertion aid 22 shown in FIG. 9 simplifies the application of the drainage foil 3. Various materials such as textiles or plastics which have low friction with respect to plastics, for example microrough textiles, can be used as insertion aid 22. The insertion aid 22 can be applied both to the tube 16 (on the left in FIG. 9) or else be integrated directly in the drainage foil 3a or be comprised in an inner lumen of the tubular drainage foil 3a (on the right in FIG. 9). For example, the insertion aid 22 can be put over a tube, for example drainage tube or a probe (on the left in FIG. 9). The tubular drainage foil 3a may have a closed tube end, for example if a tube 16 is to be sheathed with the tubular drainage foil 3a. It has been found to be advantageous for some applications if such a closed tube end is provided with a predetermined breaking point 23. By means of a predetermined breaking point 23, the insertion aid 22 can be removed by the application of force. The insertion aid 22 can also be tubular with two open tube ends (on the right in FIG. 9). In terms of production technology, it has been found to be particularly advantageous if the insertion aid 22 is already incorporated into the tubular drainage foil 3a during the production of the tubular drainage foil 3a. For simple handling of the insertion aid 22, it has proven to be favorable if the insertion aid 22 is longer than the tubular drainage foil 3a, so that it protrudes at a tube end of the tubular drainage foil 3a and can be gripped. After the tubular drainage foil 3a or a tube sheathed by a tubular drainage foil 3a has been inserted into the wound region, the insertion aid 22 can be removed by simple pulling out.

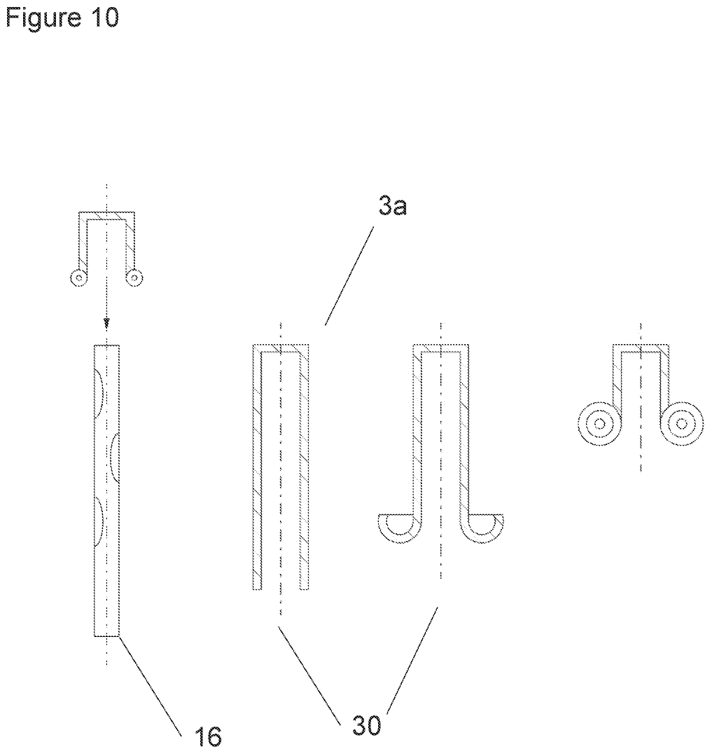

FIG. 10 illustrates rolling up the tubular drainage foil 3a in order to obtain a rolled-up tubular drainage foil 3a. Tubular drainage foils 3a rolled up in portions or completely have the advantage that they can be packaged efficiently, in particular in an environmentally friendly manner by saving material. Furthermore, they have the advantage that the user can effortlessly roll the tubular drainage foil 3a over structures, for example tubes 16, in particular drainage tubes or probes, in order to sheath them with the tubular drainage foil 3a. For rolling up the tubular drainage foil 3a, a portion of the tubular drainage foil 3a or the complete tubular drainage foil 3a can be rolled up starting from a tube opening at a tube end along a longitudinal tube axis 30. In particular, the tubular drainage foil 3a can be rolled up along the longitudinal tube axis 30 starting from an inner boundary surface in the direction of an outer boundary surface 28, in order to roll up a portion or the entire tubular drainage foil 3a.

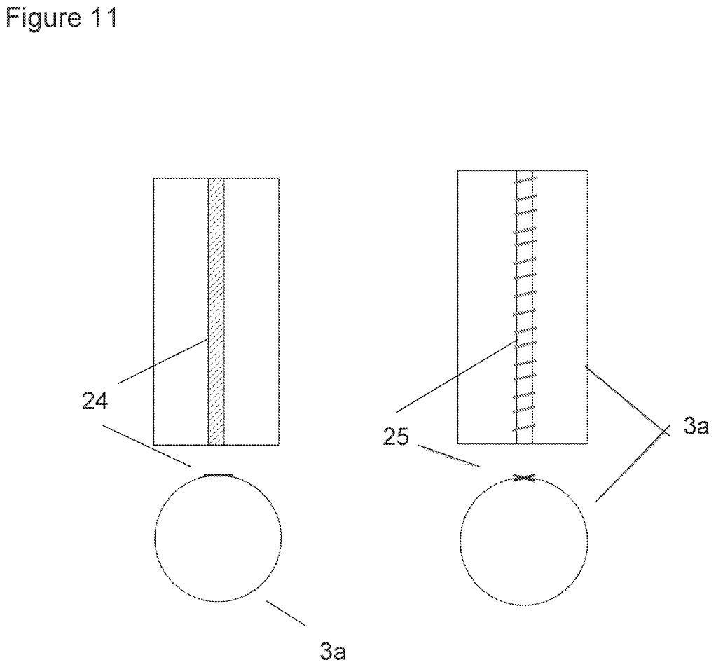

FIG. 11 shows a tubular drainage foil 3a with elastic material. The tubular drainage foil 3a can have, for example, an elastic foil 24 or an elastic seam 25, in particular a seam made of an elastic material. By integrating an elastic material into a tubular drainage foil 3a, the elasticity of the tubular drainage foil 3a can be increased, in particular if the double-walled drainage foil 3b used to form the tubular drainage foil 3a consists of a slightly expandable material. A tubular drainage foil 3a with elastic material can be formed by connecting a double-walled drainage foil 3b and an elastic material along an overlap region between the drainage foil 3b and the elastic material. By integrating the elastic material, for example a strip of elastic foil 24 or an elastic seam 25, the tubular drainage foil 3a can be expanded particularly well, as a result of which the application, for example application to a drainage tube, is facilitated.

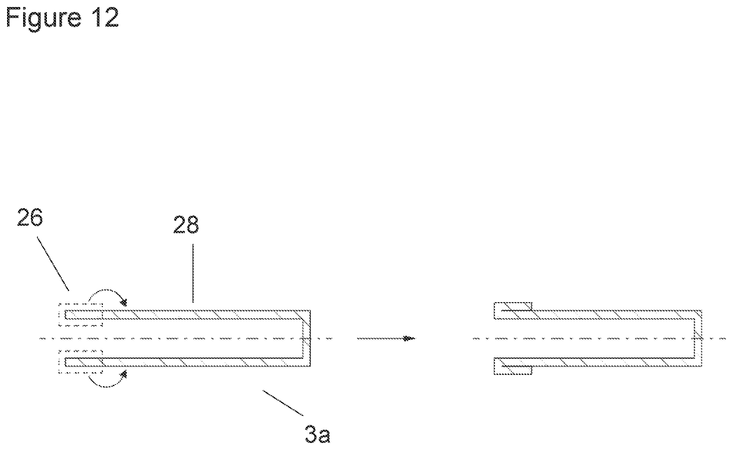

FIG. 12 illustrates a side view of a tubular drainage foil 3a with edge reinforced by folding down. The tubular drainage foil 3a can be formed at one or both tube ends in a circumferential direction with a reinforcing region. In terms of production technology, it has proven particularly efficient if the reinforcing region is formed by folding down a portion of the tubular drainage foil 3a at the tube end, wherein the folding down is carried out in particular in such a way that a tubular element forming the inner boundary surface of the tubular drainage foil 3a forms the outer boundary surface 28 of the tubular drainage foil 3a in the reinforcing region. The marked edge region 26 of the tube opening can be folded down to the outside in order thus to provide a double-walled and therefore reinforced edge of the tubular drainage foil 3a. The folded-down edge can be fixed in a punctiform manner, for example by a seam. Tearing of the tubular drainage foil 3a is effectively prevented by the reinforced edge.

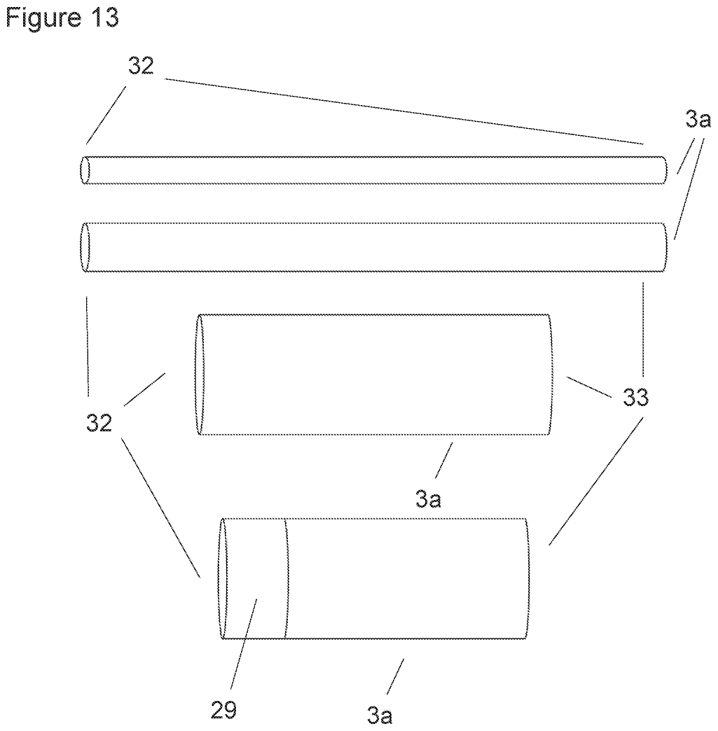

FIG. 13 illustrates various embodiments of the tubular drainage foil 3a. In particular, the tubular drainage foil 3a may have a tube opening 32 or an open tube end at one tube end and a closed tube end 33 at the other tube end. The tubular drainage foil 3a may also have a tube opening 32 at both tube ends. The tubular drainage foil 3a may furthermore have a reinforcing element 29, for example a folded-down edge.

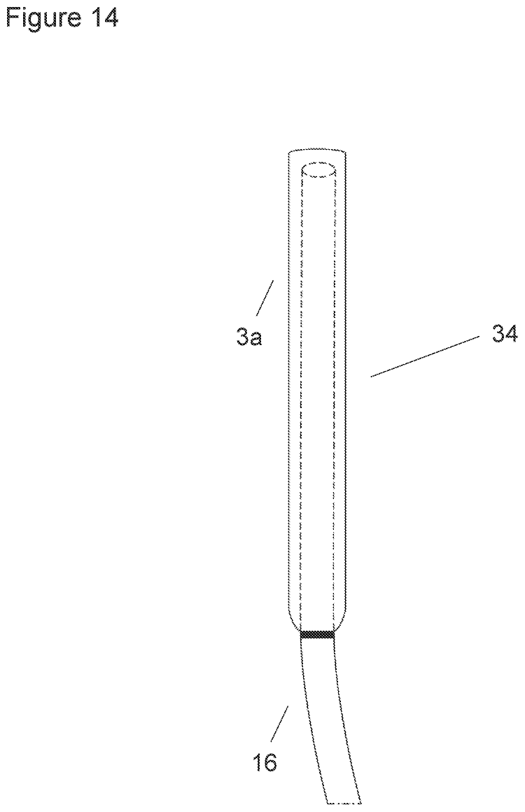

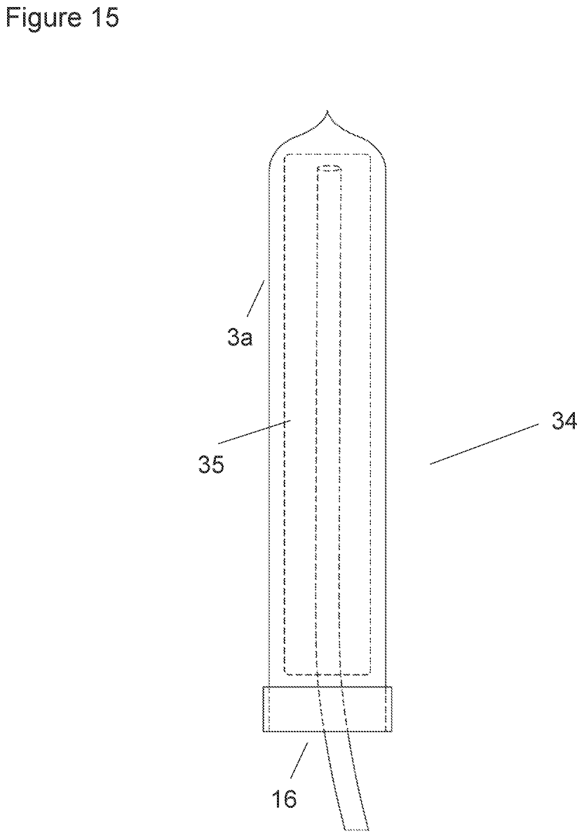

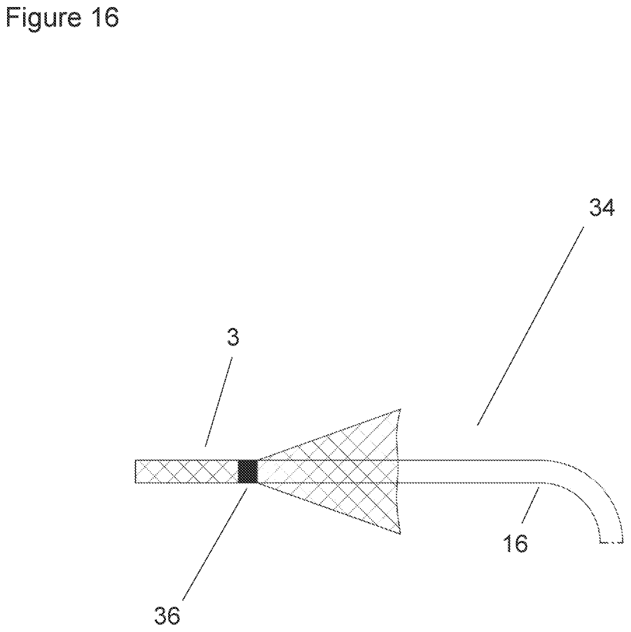

FIG. 14 shows a side view of a tubular drainage foil 3a with tube 16 which can be provided together as a wound care kit 34. FIG. 15 shows a side view of a tubular drainage foil 3a with tube 16 and absorption body 35 which can be provided together as a wound care kit 34. FIG. 16 shows a side view of a drainage foil 3, for example a tubular drainage foil 3a, with tube 16 and connecting means 36, for example an annular application aid or clamp, which can be provided together as a wound care kit 34.

Reference Signs List

| | |
|---|---|
| 1 | Welding die |
| 2 | bracket |
| 3 | Drainage foil |
| 3a | Tubular drainage foil |
| 3b | Double-walled drainage foil |
| 4 | Foundation |
| 5 | Cutting element |
| 6 | weld seam |
| 7 | Foil residue |
| 8 | ram |
| 9 | sleeve |
| 10 | side of the connecting line facing away from the fold line, |
| | in particular crease line |
| 11 | connecting line |
| 12 | side of the connecting line facing the fold line, in |
| | particular crease line |
| 13 | counter ram |
| 14 | adhesive |
| 15 | hook-and-loop fastener |
| 15a, 15b | hook-and-loop fastener |
| 16 | tube |
| 17 | first element |
| 18 | second element |
| 19 | cutting wheel |
| 20 | drainage space |
| 21 | residual material |
| 22 | insertion aid |
| 23 | predetermined breaking point |
| 24 | Elastic foil |
| 25 | Elastic seam |
| 26 | Edge region |
| 27 | Winding axis |
| 28 | Outer boundary surface of the tubular drainage foil |
| 29 | Reinforcing element |
| 30 | Tube axis |
| 31 | Inner boundary surface of the tubular drainage foil |
| 32 | Tube opening |
| 33 | Closed tube end |
| 34 | Wound care kit |
| 35 | Absorption body |
| 36 | Connecting means |

1-48. (canceled)

49. A method for producing a double-walled drainage foil with an open drainage space, wherein the method comprises:
i) providing a first web-shaped element and a second web-shaped element;
ii) perforating the first web-shaped element and the second web-shaped element, wherein in each of the first web-shaped element and the second web-shaped element, at least one opening allowing the passage of body fluids, preferably a multiplicity of openings allowing the passage of body fluids, is formed;
iii) arranging the first web-shaped element on the second web-shaped element in such a way that the first web-shaped element and the second web-shaped element are arranged approximately parallel; and
iv) connecting the first web-shaped element and the second web-shaped element at at least one fastening region, in particular at at least one punctiform fastening region, preferably by ultrasonic welding, in order to obtain a double-walled drainage foil with an open drainage space formed between the first web-shaped element and the second web-shaped element, characterized in that the method comprises forming a tubular structure from the drainage foil.

50. The method of claim 49, characterized in that the at least one opening allowing the passage of body fluids has a diameter in a range from 100 micrometers (m) to 2000 m, preferably in a range from 300 m to 700 m, more preferably in a range from 400 m to 600 m.

51. The method of claim 49, characterized in that the first web-shaped element and the second web-shaped element are connected in iv) at a multiplicity of fastening regions, preferably at a multiplicity of fastening regions formed in a grid arrangement;
wherein, preferably, a distance between in each case adjacent fastening regions is 2 millimeters (mm) or more, preferably 3 mm or more, more preferably 5 mm or more.

52. The method of claim 49, characterized in that the drainage foil has a basis weight in a range from 30 grams (g)/meter squared (m²) to 90 g/m², preferably 40 g/m² to 80 g/m², more preferably 50 g/m² to 70 g/m².

53. The method of claim 49, characterized in that the drainage foil has a tensile strength of 7 Newtons (N)/25.4 millimeters (mm) or more, an elongation at break of 40% or more, a porosity of 75 meters cubed (m³)/meter squared (m²)/minute (min) or more, and/or a bond strength of 0.5 N/25.4 mm or more.

54. The method of claim 49, characterized in that the at least one opening allowing the passage of body fluids, in particular in relation to a plan view of a planar extent of the first or second web-shaped element, has a round, circular and/or oval shape.

55. The method of claim 49, characterized in that the method comprises attaching an adhesive, in particular a glue, an adhesive material and/or a hook-and-loop fastener, in particular one or more barbs, to the drainage foil.

56. The method of claim 49, characterized in that ii) is carried out before or after iii), and/or iv) is carried out after iii) and before or after ii).

57. A method for producing a tubular drainage foil, wherein the method comprises:
a) providing the drainage foil of claim 49;
b) folding down, in particular creasing, the drainage foil onto itself about a fold line, in particular crease line, in such a way that the drainage foil has an overlap region which can be connected to form a connecting line; wherein, preferably, the drainage foil has a first edge and a second edge running substantially parallel to the first edge and the drainage foil is folded or creased onto itself along the fold line or crease line, respectively, in such a way that the drainage foil has the overlap region, which can be connected to the connecting line, along the first edge and the second edge;
c) connecting the drainage foil along the overlap region, wherein a connecting line is formed; and
d) obtaining a tubular drainage foil.

58. A method for producing a tubular drainage foil, wherein the method comprises:
a) providing the drainage foil of claim 49 and an elastic material;
b) arranging a first edge of the drainage foil on the elastic material in such a way that an overlap region, which can be connected to a connecting line, is formed between the first edge of the drainage foil and the elastic material;
c) connecting the drainage foil and the elastic material along the overlap region between the first edge of the drainage foil and the elastic material, wherein a connecting line is formed;
d) folding down, in particular creasing, the drainage foil onto the elastic material with respect to a fold line, in particular a crease line, in such a way that an overlap region, which can be connected to a connecting line, is formed between a second edge of the drainage foil, preferably a second edge of the drainage foil running substantially parallel to the first edge, and the elastic material;
e) connecting the drainage foil and the elastic material along the overlap region between the second edge of the drainage foil and the elastic material, wherein a connecting line is formed; and
f) obtaining a tubular drainage foil.

59. A method for producing a tubular drainage foil, wherein the method comprises:
a) providing the drainage foil of claim 49;
b) winding the drainage foil about a winding axis in such a way that the drainage foil has an overlap region, which is formed by the winding about the winding axis and can be connected to a connecting line and in which the drainage foil is present in at least two layers;
c) connecting a first layer and a second layer of the drainage foil present in at least two layers in the overlap region, preferably by an adhesive or a connecting means; wherein, preferably, a connecting line is formed; and
d) obtaining a tubular drainage foil.

60. The method of claim 57, wherein the connecting is carried out by welding, in particular ultrasonic welding or laser welding, by thermal bonding, in particular by means of UV or hot air, by an adhesive, in particular a glue or a hook-and-loop fastener, and/or by a connecting means, in particular a clamp.

61. The method of claim 57, wherein the connecting is carried out by ultrasonic welding, preferably at a speed in a range from 0.1 to 5.0 meters (m)/minute (min), a power in a range from 50 to 500 watts and/or a pressure in a range from 5 Newtons (N) to 100 N.

62. The method of claim 57, wherein the connecting comprises: arranging the drainage foil in an ultrasonic welding device and welding the drainage foil, in particular by means of a welding die, for example an L-shaped welding die, to form a tubular drainage foil.

63. The method of claim 57, wherein the connecting is carried out by ultrasonic welding using a cutting wheel with a cutting edge radius of 0.2 millimeters (mm) or less, and/or using a cutting wheel with a grinding angle of 15° or less facing the fold line, in particular crease line, and/or with a grinding angle of 750 or less facing away from the fold line, in particular crease line.

64. The method of claim 57, wherein an edge strip of the drainage foil arranged on a side of the connecting line facing away from the fold line, in particular crease line, is removed during the connecting or after the connecting, in particular by punching.

65. The method of claim 57, wherein the connecting line, preferably a weld seam, has a width of 2 millimeters (mm) or less, preferably 1 mm or less, more preferably 0.5 mm or less, along a circumferential direction of the tubular drainage foil.

66. The method of claim 57, wherein the connecting line, preferably a weld seam, has a thickness of 1 millimeters (mm) or less, preferably 750 micrometers (m) or less, more preferably 450 m or less, along a radial direction of the tubular drainage foil.

67. The method of claim 57, further comprising attaching a reinforcing element, in particular a reinforcing strip or a reinforcing foil, in a region of the connecting line, preferably by arranging the reinforcing element in the overlap region before the connecting, and/or at at least one tube end.

68. The method of claim 67, wherein the reinforcing element is a foil, preferably a polyethylene foil.

69. The method of claim 57, wherein the tubular drainage foil is formed at at least one tube end in a circumferential direction with a reinforcing region in which the drainage foil is present in at least two layers; and
wherein the reinforcing region is preferably formed by folding down a portion of the tubular drainage foil at the tube end, wherein the folding down is carried out in particular in such a way that a tubular element forming the inner boundary surface forms the outer boundary surface of the tubular drainage foil in the reinforcing region.

70. The method of claim 57, wherein at least one portion of the tubular drainage foil is rolled up starting from a tube opening at a tube end along a longitudinal tube axis, in particular is rolled up along a longitudinal tube axis starting from an inner boundary surface in the direction of an outer boundary surface.

71. The method of claim 57, wherein, after the connecting, a side of the connecting line facing the fold line, in particular crease line, is rotated completely outwards by inverting the tubular drainage foil.

72. A tubular drainage foil comprising:
a first tubular element forming an outer boundary surface; and
a second tubular element forming an inner boundary surface facing away from the outer boundary surface;
wherein the second tubular element is connected to the first tubular element by at least one fastening region; and
wherein the first tubular element and the second tubular element each comprise at least one opening allowing the passage of body fluids and an open drainage space is formed between the outer boundary surface and the inner boundary surface.

73. The tubular drainage foil of claim 72, wherein the first tubular element and/or the second tubular element is/are a plastic foil, and wherein the plastic foil preferably comprises polyethylene, polypropylene, polyethylene terephthalate, polyurethane, polytetrafluoroethylene, polyhydroxybutyrate, polylactide and/or cellulose.

74. The tubular drainage foil of claim 72, wherein the first tubular element and the second tubular element are connected at a multiplicity of fastening regions, preferably at a multiplicity of fastening regions formed in a grid arrangement; wherein, preferably, a distance between adjacent fastening regions is 2 mm or more, preferably 3 mm or more, more preferably 5 mm or more.

75. The tubular drainage foil of claim 72, further comprising a connecting line, preferably a weld seam, which connects the first tubular element and the second tubular element, and/or the first and/or second tubular element and an elastic material.

76. The tubular drainage foil of claim 75, characterized in that the connecting line has a width of 2 millimeters (mm) or less, preferably 1 mm or less, more preferably 0.5 mm or less, along a circumferential direction of the tubular drainage foil.

77. The tubular drainage foil of claim 75, wherein the connecting line has a thickness of 1 millimeter (mm) or less, preferably 750 micrometers (m) or less, more preferably 450 m or less, along a radial direction of the tubular drainage foil.

78. The tubular drainage foil of claim 72, characterized in that the tubular drainage foil has a basis weight in a range from 10 grams (g)/meters squared (m²) to 200 g/m², preferably 40 g/m² to 80 g/m², more preferably 50 g/m² to 70 g/m².

79. The tubular drainage foil of claim 72, wherein the tubular drainage foil has a tensile strength of 7 Newtons (N)/25.4 millimeters (mm) or more, an elongation at break of 40% or more, a porosity of 75 meters cubed (m³)/meters squared (m²)/minute (min) or more, and/or a bond strength of 0.5 N/25.4 mm or more.

80. The tubular drainage foil of claim 72, further comprising a tube opening at at least one tube end;
wherein, preferably, the tubular drainage foil has a tube opening at one tube end and has no tube opening at another tube end.

81. The tubular drainage foil of claim 72, further comprising a reinforcing element, in particular a reinforcing strip or a reinforcing foil, at at least one tube end and/or along the connecting line.

82. The tubular drainage foil of claim 72, further comprising a reinforcing region at at least one tube end in which the drainage foil is present in at least two layers;
wherein the reinforcing region is preferably formed in the form of a two-layer edge which is formed by folding down a portion of the tubular drainage foil at the tube end, wherein the folding down is carried out in particular in such a way that the second tubular element forming the inner boundary surface forms the outer boundary surface of the tubular drainage foil, in particular the outer boundary surface of the reinforcing region of the tubular drainage foil, in the reinforcing region.

83. The tubular drainage foil of claim 72, wherein the outer boundary surface, the inner boundary surface and/or an inner lumen of the tubular drainage foil comprised by the inner boundary surface comprise/comprises a sliding aid and/or an insertion aid.

84. The tubular drainage foil of claim 83, wherein the sliding aid comprises a water-based lubricant, a glycerol-based lubricant, a polymeric gel, in particular an anti-allergenic polymeric gel, an endoscopic lubricant, in particular an endoscopic sliding gel, and/or an aqueous solution, in particular a sodium chloride solution.

85. The tubular drainage foil of claim 83, wherein the insertion aid comprises a textile and/or a plastic; and/or the insertion aid is a tubular insertion aid arranged in the inner lumen of the tubular drainage foil.

86. The tubular drainage foil of claim 72, wherein the tubular drainage foil has a length in a range from 0.5 centimeters (cm) to 40 cm, preferably 2 cm to 30 cm, more preferably 5 cm to 20 cm.

87. The tubular drainage foil of claim 72, wherein the tubular drainage foil has an outer diameter in a range from 1 millimeters (mm) to 60 mm, preferably 2 mm to 50 mm, more preferably 4 mm to 30 mm.

88. A wound care kit comprising:
the tubular drainage foil of claim 72 and
at least one further constituent selected from an absorption body, a tube, a wire, a stent and an insertion aid;
wherein, preferably, at least one portion of the at least one further constituent is arranged in an inner lumen of the tubular drainage foil comprised by an inner boundary surface.

89. The wound care kit of claim 88, wherein the absorption body is a sponge, foam, in particular polyurethane or polyvinyl alcohol foam, or gauze.

90. The wound care kit claim 88, wherein the tube is a drainage tube.

91. The wound care kit of claim 88, wherein the wound care kit has a length in a range from 0.5 centimeters (cm) to 250 cm, preferably 10 cm to 200 cm.
